(19) United States
(12) Patent Application Publication
(10) Pub. No.: US 2018/0201647 A1
(43) Pub. Date: Jul. 19, 2018

(54) Peptoid

(71) Applicant: The University of Durham, Durham (GB)

(72) Inventors: Steven Cobb, Durham (GB); Hannah Bolt, Durham (GB)

(21) Appl. No.: 15/743,196

(22) PCT Filed: Jul. 27, 2016
(86) PCT No.: PCT/GB2016/052296

§ 371 Date: Jan. 9, 2018

(30) Foreign Application Priority Data
Jul. 30, 2015 (GB) 1513444.8

(51) Int. Cl. C07K 7/06; C07K 1/00; C07K 1/04; C07K 7/64; A61P 33/02; A61P 31/10; A61P 31/04

(52) U.S. Cl. CPC C07K 7/06; C07K 1/006; C07K 1/04; A61P 31/04; A61P 33/02; A61P 31/10; C07K 7/64

(57) ABSTRACT

The invention relates to peptoids, derivatives and analogues thereof, and to methods of chemically synthesising such compounds. The invention relates to mixed peptoids, derivatives and analogues thereof comprising lysine and arginine type monomers, which may be linear or cyclic, and to their uses in therapy, for example as antimicrobial agents, and in methods for treating microbial infections.

The present invention relates to peptoids, derivatives and analogues thereof, and to methods of chemically synthesising such compounds. More specifically, the present invention relates to mixed peptoids, derivatives and analogues thereof comprising lysine and arginine type monomers, which may be linear or cyclic, and to their uses in therapy, for example as antimicrobial agents, and in methods for treating microbial (e.g. bacterial) infections.

Since the discovery of penicillin in 1928, a large range of antibiotics have been successfully developed to combat a wide variety of infections. However, resistance to these antibiotics is increasing at a remarkable rate and is becoming a serious problem, with drug resistant strains of previously treatable illnesses on the rise. Current structural classes of antibiotic compounds are becoming redundant and it is widely agreed that there is a desperate need to design, make and test new antibiotic compounds.

Peptides have shown considerable promise as medicines, and investment in this area by the pharmaceutical industry continues to increase. However, many peptide drugs are readily broken down in the human body, which presents drug formulation and delivery challenges. Moreover, physical properties of peptides, such as water-solubility and membrane-permeability, remain highly problematic. Many drugs fail in development at the pre-clinical stage due to poor physical properties and many limitations remain in developing peptide-based drugs with suitable pharmaceutical properties, e.g. membrane permeability, bioavailability and water solubility. Aqueous formulation of peptides can, therefore, be non-trivial and are often formulated with excipients, surfactants and co-solvents, which may result in adverse side effects. Therefore, there is a need to improve peptidic drugs due to their inherently unfavourable pharmacokinetic properties, e.g. stability, membrane permeability, bioavailability and water solubility.

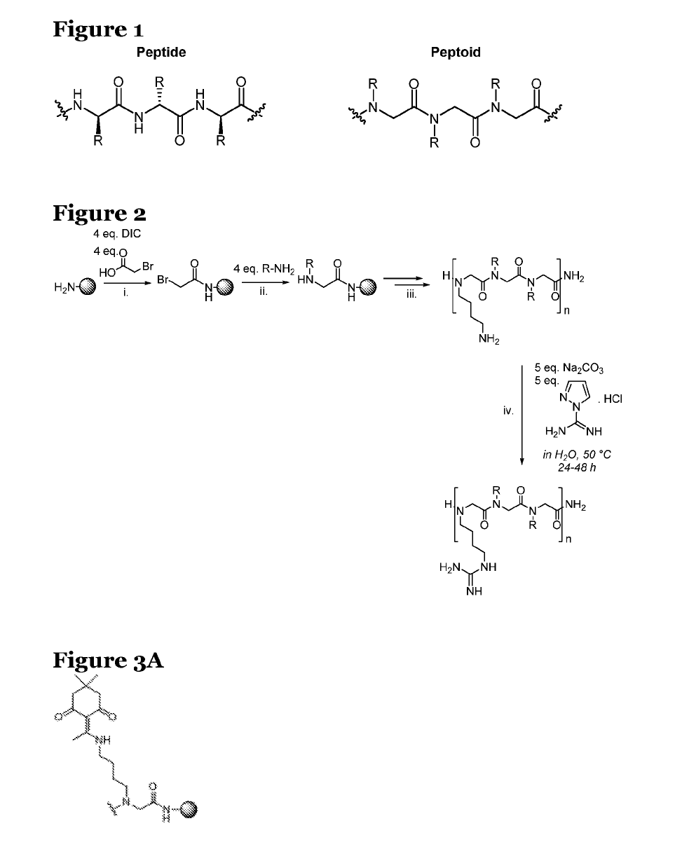

In the development of more stable peptide drugs areas of significant interest lie in the use of stabilised or stapled α-helices, (multi)cyclic peptides and peptidomimetics, which include ‘peptoids’. Peptoids, or poly-N-substituted glycines, are a class of peptidomimetics whose side chains are appended to the nitrogen atom of the peptide backbone, rather than to the alpha-carbons, as they are in amino acids, and this is shown in FIG. 1. Peptoids are an emerging class of therapeutic agent, which are structurally very similar to peptides, but have a superior proteolytic stability in vivo when compared with standard peptide-based drugs.

Commonly, lysine-containing peptoids are seen in the literature. Some research groups have sought to improve the biological activity of peptoids by replacing these lysine′ residues with the ‘arginine’ analogue (a primary amine to a guanidinium group) since arginine-rich cell-penetrating peptides have been shown to have a high potential to deliver drugs into cultured cells. Guanidine-containing peptoids have been shown to translocate into the cell quicker than amino containing peptoids. [P. A. Wender, D. J. Mitchell, K. Pattabiraman et al., Proc. Natl. Acad. Sci. USA, 2000, 97, 13003-13008; M. L. Huang, S. B. Y. Shin, M. A. Benson et al., ChemMedChem, 2012, 7, 114-122.]

Previously synthesised polyarginine-based peptoids were made using the method developed by Rothbard and co-workers using pyrazole-1-carboxamide to modify lysine type side-chains after peptoid synthesis and cleavage from the resin, and shown in FIG. 2. [P. A. Wender, D. J. Mitchell, K. Pattabiraman et al., Proc. Natl. Acad. Sci. USA, 2000, 97, 13003-13008]. The disadvantage of this procedure however is that every lysine-type chain is transformed into an arginine-type chain and so mixed peptoid sequences comprising both lysines and arginines cannot be made. This approach only makes arginine side chain peptoids, and mixed Arg/Lys peptoids cannot be accessed using this approach.

Currently there are challenges in balancing the biological activity and the toxicity of peptoid compounds. The inventors believe that the ability to alter the chemical functionality of the cationic side chains in a given peptoid sequence, such as the lysine and arginine type monomers, may assist in these endeavours.

The Zuckermann group (http://www.ronznet.com/index.html) has previously described the synthesis of a PMC-protected guanidinopropyl amine that was suggested to be compatible with the submonomer synthesis of peptoids on resin (T. Uno, E. Beausoleil, R. A. Goldsmith et al., Tetrahedron Lett., 1999, 40, 1475-1478). Barron et al., attempted the synthesis of a mixed Arg/Lys peptoid using these PMC-protected amines, however, their poor solubility and the extended cleavage times necessary caused acid-induced degradation of the mixed peptoids and prevented isolation of the required targets (S. L. Seurynck-Servoss, M. T. Dohm and A. E. Barron, Biochem., 2006, 45, 11809-11818). It was only possible to prepare and isolate one mixed Arg/Lys peptoid which was linear and contained no aromatic residues.

Aromatic residues/side chains (particularly α-chiral aromatic residues) have been shown to stabilise a helical secondary structure, which can be important for the biological or materials applications of peptoids. Additionally, being able to include aromatic residues vastly increases the possible sequence variety.

It would be advantageous to be able to be able to prepare mixed Arg/Lys type peptoids (i.e. peptoids that contain amine and guanidine functionalities on their side chains, similar to the generic peptoid structure shown in FIG. 12) that were cyclic and/or contain aromatic side chains.

The current invention arises from the inventors' work in trying to overcome the problems associated with the prior art.

In accordance with a first aspect of the invention, there is provided a method of preparing a peptoid, analogue or derivative thereof, comprising at least one arginine type monomer and at least one lysine type monomer, the method comprising:—
(i) synthesising a precursor linear peptoid, analogue or derivative thereof comprising one or more lysine type monomers protected with a first protecting group, and one or more lysine type monomers protected with a second protecting group, wherein the first and second protecting groups are orthogonal;
(ii) removing the first protecting group to reveal one or more unprotected lysine type monomers;
(iii) converting the one or more unprotected lysine type monomers to one or more arginine type monomers; and
(iv) removing the second protecting group to obtain a peptoid, analogue or derivative thereof, comprising at least one arginine type monomer and at least one lysine type monomer.

It will be appreciated that the term “arginine type monomer” can refer to a monomer with guanidine functionality. Accordingly, the term “arginine type monomer” may refer to a monomer of Formula (I):

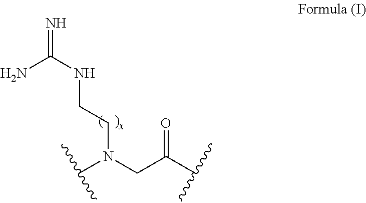

wherein x is an integer between 0 and 14.

Preferably, x is an integer between 0 and 9. More preferably, x is an integer between 0 and 5. Accordingly, x may be 0, 1, 2, 3, 4 or 5.

Accordingly, when x is 2, the arginine type monomer comprises an N-(3-guanidinopropyl) glycine (NArg) monomer. Alternatively when x is 3 the arginine type monomer comprises N-(4-guanidinobutyl) glycine (NhArg) monomer, and when x is 1 the arginine type monomer comprises an N-(2-guanidinoethyl) glycine (NnArg) monomer.

It will be appreciated that the term “lysine type monomer” can refer to a monomer with amine functionality. Accordingly, the term “lysine type monomer” may refer to a monomer of Formula (II):

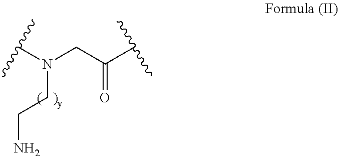

wherein y is an integer between 0 and 14.

Preferably, y is an integer between 0 and 9. More preferably, y is an integer between 0 and 5. Accordingly, y may be 0, 1, 2, 3, 4 or 5.

Accordingly, when y is 1, the lysine type monomer comprises an N-(2-aminoethyl) glycine (Nae) monomer. When y is 3, the lysine type monomer comprises an N-(4-aminobutyl) glycine (NLys) monomer. When y is 5, the lysine type monomer comprises an N-(6-aminohexyl) glycine (Nah) monomer.

Advantageously, the method enables the synthesis of a peptoid, derivative or analogue thereof comprising at least one lysine type monomer and at least one arginine type monomer.

It will be appreciated that “orthogonal protection” is a strategy allowing the deprotection of multiple protective groups one at a time, each with a dedicated set of reaction conditions, without affecting the other. Thus, the term “orthogonal” can mean that the first protecting group can be removed from its lysine type monomer under conditions which do not cause the second protecting group to be removed from its corresponding lysine type monomer. Hence, deprotection of the first lysine type monomer is independent from deprotection of the second lysine type monomer.

The term “derivative or analogue thereof” can mean that the amino acids residues of the peptoid are replaced by residues (whether natural amino acids, non-natural amino acids or amino acid mimics) with similar side chains or peptoid backbone properties. Additionally, the terminals of such peptoids may be protected by N- and C-terminal protecting groups with similar properties to acetyl or amide groups.

Derivatives and analogues of peptoids according to the invention may also include retropeptoid derivatives. A retropeptoid is expected to bind in the opposite direction in the ligand-binding groove, as compared to a peptide or peptoid-peptide hybrid containing one peptoid residue. As a result, the side chains of the peptoid residues are able point in the same direction as the side chains in the original peptide. Peptoid-peptide hybrids are also envisaged as derivatives or analogues described herein.

The first and second protecting groups may comprise an N-(1-(4,4-dimethyl-2,6-dioxocyclohexylidene)ethyl) (Dde) protecting group, a tert-Butyloxycarbonyl (Boc) protecting group, a pentamethyl-2,3-dihydrobenzofuran-5-sulfonyl (Pbf) protecting group, a ω,ω′-bis-Allyloxycarbonyl (Alloc) protecting group, a 2,2,5,7,8-pentamethylchroman-6-sulfonyl (Pmc) protecting group, a trityl (Trt) protecting group, a t-butyl ester (OtBu) protecting group, a 4-Methyltrityl (Mtt) protecting group, a ω,ω′-bis-benzyloxycarbonyl (Z) protecting group, and/or a benzyl (Bzl) protecting group. In a preferred embodiment, the first protecting group comprises an N-(1-(4,4-dimethyl-2,6-dioxocyclohexylidene)ethyl) (Dde) protecting group and the second protecting group comprises a tert-Butyloxycarbonyl (Boc) protecting group.

Preferably, the step of synthesising the linear precursor peptoid, analogue or derivative thereof comprises synthesising a linear precursor peptoid, analogue or derivative thereof on a substrate surface in a step-wise fashion.

The step of synthesising the linear precursor peptoid, analogue or derivative thereof may comprise:
synthesising a first monomer on a substrate to obtain a linear precursor peptoid, analogue or derivative thereof comprising one monomer;
subsequently adding at least one further monomer in a step wise fashion to obtain the linear precursor peptoid, analogue or derivative thereof containing the desired number of monomers.

It will be appreciated that the first and/or at least one further monomers may comprise at least one lysine type monomer protected with a first protecting group and at least one lysine type monomer protected with a second protecting group. Additionally, the first and/or at least one further monomers may comprise at least one monomer comprising an aromatic residue and/or at least one monomer comprising an aliphatic residue.

The monomer comprising an aromatic residue may comprise an (S)—N-(1-phenylethyl) glycine (Nspe) monomer, an (R)—N-(1-phenylethyl) glycine (Nrpe) monomer, an N-(phenylmethyl) glycine (Nphe) monomer, an N-(4-fluoro phenylmethyl) glycine (Npfb) monomer, an N-(3-fluoro phenylmethyl) glycine (Nmfb) monomer, an (S)—N-1-(4-fluoro phenylethyl) glycine (Nsfb) monomer, an (R)—N-1-(4-fluoro phenylethyl) glycine (Nrfb) monomer, an N-(3,5 difluoro phenylmethyl) glycine (Ndfb) monomer, an N-(4-chloro phenylmethyl) glycine (Npcb) monomer, an N-(4-methoxyphenylmethyl) glycine (Npmb) monomer, an N-(methylimidazole) glycine (NHis) monomer, an N-(methylindole) glycine (NTrp) monomer, an N-(4-hydroxy phenylmethyl) glycine (NTyr) monomer, an N-(4-pyridinylmethyl) glycine (NPyr) monomer, an (S)—N-(1-naphthlethyl) glycine (Nsna) monomer, an (R)—N-(1-naphthlethyl) glycine (Nrna) monomer, an N-(furanylmethyl) glycine (Nfur) monomer, an N-(thiofuranylmethyl) glycine (Ntfur) monomer or an N-(diphenylmethyl) glycine (Ndpa) monomer.

Preferably, the monomer comprises an Nspe monomer or an Nrpe monomer.

The monomer comprising an aliphatic residue may comprise an N-(pentyl) glycine (Namy) monomer, an N-(propyl) glycine (NNVa) monomer, an N-(isopentyl) glycine (NHLe) monomer, N-(isobutyl) glycine (NLeu) monomer, an N-(butyl) glycine (Nbut) monomer, an N-(2-carboxyethyl) glycine (NGlu) monomer, an N-(2,2,2-trifluoromethyl) glycine (Ntfe) monomer, an N-(2,2,3,3,3-pentafluoropropyl) glycine (Npfp), an N-(2,2-difluoroethyl) glycine (Ndfea) monomer, an N-(ethyl) glycine (Nea) monomer, an N-(2-thioethyl) glycine (NCys) monomer, an (S)—N-(sec-butyl) glycine (Nssb) monomer, an (R)—N-(sec-butyl) glycine (Nrsb) monomer, an (S)—N-(1-methylbutyl) glycine (Nsmb) monomer, an (R)—N-(1-methylbutyl) glycine (Nrmb) monomer, an (S)—N-(1-cyclohexylethyl) glycine (Nsch) monomer, (R)—N-(1-cyclohexylethyl) glycine (Nrch) monomer, an N-(1-cyclohexylmethyl) glycine (Nch) monomer, an N-(ethynylmethyl) glycine (Nem) monomer, an (S)—N-(1-ethynylethyl) glycine (Nsee) monomer, or an (R)—N-(1-ethynylethyl) glycine (Nree) monomer.

Accordingly, the precursor linear peptoid, analogue or derivative thereof may comprise one or more aliphatic and/or aromatic residues. The aliphatic and/or aromatic residues may be disposed at or towards the N-terminus, at or towards the C-terminus, or within the core of the peptoid, analogue or derivative thereof.

Advantageously, the method enables the synthesis of a peptoid, analogue or derivative thereof comprising lysine, arginine and aromatic residues.

The substrate preferably comprises a resin. The resin may comprise Rink amide resin, 2-chlorotrityl chloride resin or Wang resin, 4-(1′,1′-dimethyl-1′-hydroxypropyl) phenoxyacetyl alanyl aminomethyl polystyrene (DHPP) resin or diphenyldiazomethane (PDDM) resin.

The step of synthesising a first monomer comprising on a substrate may comprise:
contacting a substrate with haloacetic acid; and
contacting the substrate with a desired amine sub-monomer to obtain a linear precursor peptoid, analogue or derivative thereof comprising one monomer.

The step of subsequently adding a further monomer may comprise:
contacting the substrate with haloacetic acid; and
contacting the substrate with a desired amine sub-monomer.

The step of subsequently adding a further monomer may be repeated until the linear precursor peptoid, analogue or derivative thereof contains the desired number of monomers.

The halo acetic acid preferably comprises bromoacetic acid.

Prior to the step of contacting the substrate with haloacetic acid, the method may comprise a step of contracting the substrate with a solvent. Preferably, the step of contacting the substrate with the solvent lasts for at least one hour. More preferably, the step of contacting the substrate with the solvent lasts for at least two, three, four or five hours. Most preferably, the step of contacting the substrate with the solvent lasts for at least six, seven, eight, nine or ten hours.

Preferably, the step of contacting the substrate with the solvent is undertaken at about room temperature.

Preferably, the solvent is dimethyl formamide (DMF), dichloromethane (DCM), dimethylacetamide (DMA) or N-methyl-2-pyrrolidone (NMP). Preferably, in embodiments where the substrate comprises Rink amide resin the solvent comprises dimethyl formamide (DMF). Preferably, in embodiments where the substrate comprises 2-chlorotrityl chloride resin the solvent comprises dichloromethane (DCM).

Preferably, the step of contacting the substrate with haloacetic acid is undertaken in the presence of a base. Preferably, the base if N,N′-diisopropylcarbodiimide (DIC) or N,N-Diisopropylethylamine (DIPEA).

Preferably, the step of contacting the substrate with haloacetic acid lasts for at least 5 minutes. More preferably, the step of contacting the substrate with haloacetic acid lasts for at least 10 or 15 minutes. Most preferably, the step of contacting the substrate with haloacetic acid lasts for at least 20 minutes.

Preferably, the step of contacting the substrate with haloacetic acid is undertaken at about room temperature.

Preferably, the molar ratio of the base to the substrate is at least 1:1. More preferably, the molar ratio of the base to the substrate is at least 2:1, 3:1, 4:1, 5:1, 6:1 or 7:1. Most preferably, the molar ratio of the base to the substrate is at least 8:1.

Preferably, the molar ratio of the haloacetic acid to the substrate is at least 1:1. More preferably, the molar ratio of the haloacetic acid to the substrate is at least 2:1, 3:1, 4:1, 5:1, 6:1 or 7:1. Most preferably, the molar ratio of the haloacetic acid to the substrate is at least 8:1.

Preferably, the step of contacting the substrate with the desired amine sub-monomer lasts for at least 5 minutes. More preferably, the step of contacting the substrate with the desired amine sub-monomer lasts for at least 10, 15, 20, 25 or 30 minutes. Most preferably, the step of contacting the substrate with the desired amine sub-monomer lasts for at least 35, 40, 45, 50, 55 or 60 minutes.

Preferably, the step of contacting the substrate with the desired amine sub-monomer is undertaken at about room temperature.

Preferably, the molar ratio of the desired amine sub-monomer to the substrate is at least 1:1. More preferably, the molar ratio of the desired amine sub-monomer to the substrate is at least 2:1, 3:1, 4:1, 5:1, 6:1 or 7:1. Most preferably, the molar ratio of the desired amine sub-monomer to the substrate is at least 8:1.

The desired amine sub-monomer may comprise a C₁₋₁₅ alkane substituted with an unprotected amino group on each terminal carbon, to obtain an unprotected lysine type monomer on the substrate.

Alternatively, the desired amine sub-monomer may comprise a C₁₋₁₅ alkane substituted with an unprotected amino group on a first terminal carbon and a protected amino group on a second terminal carbon, to obtain a protected lysine type monomer on the substrate. The protected amino group may be protected by the first protecting group or the second protecting group. Preferably, the protected amino group is protected by the Boc protecting group.

The C₁₋₁₅ straight chain alkane may comprise 1,2 diaminoethane, 1,3 diaminopropane, 1,4 diaminobutane, 1,5 diaminopentane, 1,6 diaminohexane, 1,7 diaminoheptane, 1,8-diaminooctane, 1,9-diaminononane, 1,10-diaminodecane, 1,11-diaminoundecane, 1,12-diaminododecane, 1,13-diaminotridecane, 1,14-diaminotetradecane or 1,15-diaminopentadecane.

In embodiments where the monomer comprises an unprotected lysine type monomer the method may comprise an additional step carried out subsequent to contacting the substrate with the C₁₋₁₅ alkane, the subsequent step comprising contacting the unprotected lysine type monomer with a first further reagent.

Preferably, the step of contacting the unprotected lysine type monomer with the first further reagent lasts for at least 5 minutes. More preferably, the step of contacting the unprotected lysine type monomer with the first further reagent lasts for at least 10, 20, 30, 40, 50 or 60 minutes. Most preferably, the step of contacting the unprotected lysine type monomer with the first further reagent lasts for at least 70, 80, 90, 100, 110 or 120 minutes.

Preferably, the molar ratio of the first further reagent to the unprotected lysine type monomer is at least 1:1. More preferably, the first further reagent to the unprotected lysine type monomer is at least 2:1, 3:1, 4:1, 5:1, 6:1, 7:1, 8:1 or 9:1. Most preferably, the first further reagent to the unprotected lysine type monomer is at least 10:1.

Preferably, the step of contacting the unprotected lysine type monomer on the substrate with the first further reagent is undertaken at about room temperature.

Preferably, the first further reagent comprises 2-acetyldimedone (Dde-OH), di-tert-butyl dicarbonate ((Boc)₂O), 2,2,4,6,7-Pentamethyldihydrobenzofuran-5-sulfonyl chloride (Pbf-Cl), an alloc protecting group introducing reagent, 2,2,5,7,8-Pentamethyl-chromane-6-sulfonyl chloride (Pmc-Cl), [chloro-di(phenyl)methyl]benzene (Trt-Cl), t-Butyl Alcohol (tBuOH), tosyl chloride (Ts-Cl), tert-butyldimethylsilyl-chloride (TBDMS-Cl), Methoxytriphenylmethyl chloride (MMT-Cl), a Benzoyl (Z) protecting group introducing agent and a Benzyl (Bn) protecting group introducing agent. Most preferably, the first further reagent comprises 2-acetyldimedone (Dde-OH).

Alternatively, the desired amine sub-monomer may comprise a molecule comprise an amine and an aromatic or aliphatic group configured to obtain a monomer comprising an aromatic residue or aliphatic residue as defined above.

In embodiments where the first protecting group is Dde the step of removing the first protecting group to reveal one or more unprotected lysine type monomers may comprise contacting the peptoid, analogue or derivative thereof with hydrazine.

In embodiments where the second protecting group is Dde the step of removing the second protecting group to reveal one or more unprotected lysine type monomers may comprise contacting the peptoid, analogue or derivative thereof with hydrazine.

Preferably, the step of contacting the peptoid, analogue or derivative thereof with hydrazine lasts for at least 1 minutes. More preferably, the step of contacting the peptoid, analogue or derivative thereof with hydrazine lasts for at least 2 minutes. Most preferably, the step of contacting the peptoid, analogue or derivative thereof with hydrazine lasts for at least 3 minutes.

Preferably, the step of contacting the peptoid, analogue or derivative thereof with hydrazine is repeated at least once. More preferably, the step of contacting the peptoid, analogue or derivative thereof with hydrazine is repeated at least two or three times. Most preferably, the step of contacting the peptoid, analogue or derivative thereof with hydrazine is repeated at least four times.

Preferably, the step of contacting the peptoid, analogue or derivative thereof with hydrazine is undertaken at about room temperature.

In embodiments where the first protecting group is a tert-Butyloxycarbonyl (Boc) protecting group the step of removing the first protecting group to reveal one or more unprotected lysine type monomers may comprise contacting the peptoid, analogue or derivative thereof with a deprotecting solution comprising trifluoroacetic acid (TFA), water and/or triisopropylsilane (TIPS). Preferably, the step of removing the first protecting group to reveal one or more unprotected lysine type monomers comprises contacting the peptoid, analogue or derivative thereof with a deprotecting solution comprising trifluoroacetic acid (TFA), water and triisopropylsilane (TIPS).

In embodiments where the second protecting group is a tert-Butyloxycarbonyl (Boc) protecting group the step of removing the second protecting group to reveal one or more unprotected lysine type monomers may comprise contacting the peptoid, analogue or derivative thereof with a deprotecting solution comprising trifluoroacetic acid (TFA), water and/or triisopropylsilane (TIPS). Preferably, the step of removing the second protecting group to reveal one or more unprotected lysine type monomers comprises contacting the peptoid, analogue or derivative thereof with a deprotecting solution comprising trifluoroacetic acid (TFA), water and triisopropylsilane (TIPS).

Preferably, the deprotecting solution comprises at least 50% (v/v) trifluoroacetic acid (TFA). More preferably, the deprotecting solution comprises at least 60% (v/v), 70% (v/v), 80% (v/v) or 90% (v/v) trifluoroacetic acid (TFA). Most preferably, the deprotecting solution comprises about 95% (v/v) trifluoroacetic acid (TFA).

Preferably, the deprotecting solution comprises at least 0.5% (v/v) water. More preferably, the deprotecting solution comprises at least 1.0% (v/v), 1.5% (v/v) or 2.0% (v/v) water. Most preferably, the deprotecting solution comprises about 2.5% (v/v) water.

Preferably, the deprotecting solution comprises at least 0.5% (v/v) triisopropylsilane (TIPS). More preferably, the deprotecting solution comprises at least 1.0% (v/v), 1.5% (v/v) or 2.0% (v/v) triisopropylsilane (TIPS). Most preferably, the deprotecting solution comprises about 2.5% (v/v) triisopropylsilane (TIPS).

In a most preferred embodiment, the deprotecting solution comprises about 95% (v/v) trifluoroacetic acid (TFA), about 2.5% (v/v) water and about 2.5% (v/v) triisopropylsilane (TIPS).

Preferably, the step of contacting the peptoid, analogue or derivative thereof with a deprotecting solution lasts for at least 5 minutes. More preferably, the step of contacting the peptoid, analogue or derivative thereof with a deprotecting solution lasts for at least 10, 20, 30, 40 or 50 minutes. Most preferably, the step of contacting the peptoid, analogue or derivative thereof with a cleavage solution lasts for at least 60, 70, 80 or 90 minutes.

Preferably, the step of contacting the peptoid, analogue or derivative thereof with the deprotecting solution is undertaken at about room temperature.

The step of converting the one or more unprotected lysine type monomers to one or more arginine type monomers may comprise contacting the or each unprotected lysine type monomers with pyrazole-1-carboxamide. Preferably, the molar ratio of pyrazole-1-carboxamide to the or each unprotected lysine type monomer is at least 1:1. More preferably, the molar ratio of pyrazole-1-carboxamide to the or each unprotected lysine type monomer is at least 2:1, 3:1, 4:1 or 5:1. Most preferably, the molar ratio of pyrazole-1-carboxamide to the or each unprotected lysine type monomer is at least 6:1.

Preferably, the unprotected lysine type monomers are contacted with pyrazole-1-carboxamide in the presence of a base. Preferably, the base comprises diisopropylethylamine (DIPEA), triethylamine (TEA) or N-Methylmorpholine (NMM). Most preferably, the base comprises diisopropylethylamine (DIPEA). Preferably, the molar ratio of base to the or each unprotected lysine type monomer is at least 1:1. More preferably, the molar ratio of base to the or each unprotected lysine type monomer is at least 2:1, 3:1, 4:1 or 5:1. Most preferably, the molar ratio of base to the or each unprotected lysine type monomer is at least 6:1.

Preferably, the step of contacting the unprotected lysine type monomers with pyrazole-1-carboxamide lasts for at least 5 minutes. More preferably, the step of contacting the unprotected lysine type monomers with pyrazole-1-carboxamide lasts for at least 10, 20, 30, 40 or 50 minutes. Most preferably, the step of contacting the unprotected lysine type monomers with pyrazole-1-carboxamide lasts for at least 60, 70 or 80 minutes.

Preferably, the step of contacting the unprotected lysine type monomers with pyrazole-1-carboxamide is undertaken at about room temperature.

Preferably, the method comprises a step of cleaving the peptoid, derivative or analogue thereof from the substrate to obtain a cleaved precursor linear peptoid, analogue or derivative thereof.

The step of cleaving the substrate may comprise contacting the peptoid, derivative or analogue thereof with a cleavage solution. The cleavage solution may comprise trifluoroacetic acid (TFA), water, triisopropylsilane (TIPS), dichloromethane (DCM), acetic acid and/or hexafluoro-2-propanol (HFIP).

In one embodiment, the cleavage solution comprises trifluoroacetic acid (TFA).

The cleavage solution may comprise about 100% trifluoroacetic acid (TFA).

Alternatively, the cleavage solution may comprise trifluoroacetic acid (TFA) and dichloromethane (DCM).

Alternatively, the cleavage solution comprises trifluoroacetic acid (TFA), water and triisopropylsilane (TIPS). Preferably, in this embodiment, the substrate comprises Rink amide resin.

Preferably, the cleavage solution comprises at least 50% (v/v) trifluoroacetic acid (TFA). More preferably, the cleavage solution comprises at least 60% (v/v), 70% (v/v), 80% (v/v) or 90% (v/v) trifluoroacetic acid (TFA). Most preferably, the cleavage solution comprises about 95% (v/v) trifluoroacetic acid (TFA).

Preferably, the cleavage solution comprises at least 0.5% (v/v) water. More preferably, the cleavage solution comprises at least 1.0% (v/v), 1.5% (v/v) or 2.0% (v/v) water. Most preferably, the cleavage solution comprises about 2.5% (v/v) water.

Preferably, the cleavage solution comprises at least 0.5% (v/v) triisopropylsilane (TIPS). More preferably, the cleavage solution comprises at least 1.0% (v/v), 1.5% (v/v) or 2.0% (v/v) triisopropylsilane (TIPS). Most preferably, the cleavage solution comprises about 2.5% (v/v) triisopropylsilane (TIPS).

In a most preferred embodiment, the cleavage solution comprises about 95% (v/v) trifluoroacetic acid (TFA), about 2.5% (v/v) water and about 2.5% (v/v) triisopropylsilane (TIPS).

In an alternative embodiment, the cleavage solution comprises acetic acid.

In a further alternative embodiment, the cleavage solution comprises hexafluoro-2-propanol (HFIP). Preferably, in this embodiment the substrate comprises 2-chlorotrityl chloride resin.

Preferably, the step of contacting the peptoid, derivative or analogue thereof with a cleavage solution lasts for at least 5 minutes. More preferably, the step of contacting the peptoid, derivative or analogue thereof with a cleavage solution lasts for at least 10, 15, 20 or 25 minutes. Most preferably, the step of contacting the peptoid, derivative or analogue thereof with a cleavage solution lasts for at least 30 minutes.

Preferably, the step of contacting the peptoid, derivative or analogue thereof with a cleavage solution is undertaken at about room temperature.

Preferably, prior to the step of contacting the peptoid, derivative or analogue thereof with a cleavage solution the method comprises shrinking the substrate. Preferably, the step of shrinking the substrate comprises contacting the resin with ether.

In one embodiment, the cleaving step is carried out subsequent to step (i) and prior to step (ii). Accordingly, the precursor linear peptoid, derivative or analogue thereof may be cleaved from the substrate prior to the steps of removing either the first or second protecting groups. Preferably, the substrate comprises 2-chlorotrityl chloride resin. Preferably, the first and second protecting groups comprise Dde and a tert-Butyloxycarbonyl (Boc) protecting group.

Advantageously, when the substrate comprises 2-chlorotrityl chloride resin and the protecting groups comprise Dde and a tert-Butyloxycarbonyl (Boc) protecting group it is possible to cleave the precursor linear peptoid, derivative or analogue thereof from the substrate without removing either of the protecting groups.

The peptoid, analogue or derivative thereof, comprising at least one arginine type monomer and at least one lysine type monomer may comprise a linear peptoid, derivative or analogue thereof. Accordingly, step (ii) may be carried out on the cleaved precursor linear peptoid, analogue or derivative thereof.

Alternatively, the peptoid, analogue or derivative thereof, comprising at least one arginine type monomer and at least one lysine type monomer may comprise a cyclic peptoid, derivative or analogue thereof.

Accordingly, the method may comprise a cyclisation step comprising cyclising the precursor linear peptoid, derivative or analogue thereof to obtain a precursor cyclic peptoid. The cyclisation step may be carried out subsequent to the cleaving step. The cyclisation step may be carried out prior to step (ii). Accordingly, step (ii) may be carried out on the precursor cyclic peptoid, derivative or analogue thereof.

Advantageously, this will allow a user to synthesise a cyclic peptoid, derivative or analogue thereof comprising lysine and arginine type monomers.

The cyclisation step may comprise contacting the cleaved precursor linear peptoid, analogue or derivative thereof with a coupling reagent. The coupling reagent may comprise benzotriazol-1-yl-oxytripyrrolidinophosphonium hexafluorophosphate (PyBOP), N,N′-diisopropylcarbodiimide (DIC), N,N′-dicyclohexylcarbodiimide (DCC), ethyl (hydroxyimino)cyanoacetate (Oxyma) or O-(benzotriazol-1-yl)-N,N,N′,N′-tetramethyluronium hexafluorophosphate (HBTU). Preferably, the molar ratio of coupling reagent to the cleaved precursor linear peptoid, analogue or derivative thereof at least 1:1. More preferably, the molar ratio of coupling reagent to the cleaved precursor linear peptoid, analogue or derivative thereof is at least 2:1, 3:1, 4:1 or 5:1. Most preferably, the molar ratio of coupling reagent to the cleaved precursor linear peptoid, analogue or derivative thereof is at least 6:1.

The step of contacting the cleaved precursor linear peptoid, analogue or derivative thereof with coupling reagent may be conducted in the presence of a base. The base may comprise N,N-Diisopropylethylamine (DIPEA), triethylamine (TEA) or N-Methylmorpholine (NMM). Preferably, the base comprises N,N-Diisopropylethylamine (DIPEA). Preferably, the molar ratio of the base to the cleaved precursor linear peptoid, analogue or derivative thereof is at least 1:1. More preferably, the molar ratio of the base to the cleaved precursor linear peptoid, analogue or derivative thereof is at least 2:1, 3:1, 4:1 or 5:1. Most preferably, the molar ratio of the base to the cleaved precursor linear peptoid, analogue or derivative thereof is at least 6:1.

Preferably, the step of contacting the cleaved precursor linear peptoid, analogue or derivative thereof with coupling reagent lasts for at least 5 minutes. More preferably, the step of contacting the cleaved precursor linear peptoid, analogue or derivative thereof with coupling reagent lasts for at least 10, 20, 30, 40 or 50 minutes. Most preferably, the step of contacting the cleaved precursor linear peptoid, analogue or derivative thereof with coupling reagent lasts for at least 60 minutes.

Preferably, the step of contacting the cleaved precursor linear peptoid, analogue or derivative thereof with coupling reagent is undertaken at about room temperature.

In an alternative embodiment, the cleaving step may be carried out subsequent to step (iii). Accordingly, the cleaving step may be carried out prior to the step of removing the second protecting group. Alternatively, the cleaving step may be carried out subsequent to the step of removing the second protecting group. However, in a preferred embodiment the cleaving step is carried out simultaneously to the step of removing the second protecting group. In this embodiment the substrate may comprise Rink amide resin and the second protecting group may comprise a tert-Butyloxycarbonyl (Boc) protecting group.

Advantageously, in embodiments where the substrate comprises Rink amide resin and the second protecting group comprises a tert-Butyloxycarbonyl (Boc) protecting group the conditions used to remove the second protecting group also cleave the peptoid, derivative or analogue thereof from the substrate. Advantageously, by cleaving the peptoid, derivative or analogue thereof from the substrate at the same time as removing the second protecting group, a linear peptoid, derivative or analogue thereof is obtained.

The inventors believe that the peptoids which may be obtained using the above method are novel per se.

Hence, in accordance with a second aspect, there is provided a peptoid, analogue or derivative thereof, comprising at least one arginine type monomer, at least one lysine type monomer and at least one monomer comprising an aromatic residue.

Preferably, the peptoid, analogue or derivative thereof is obtained using the method of the first aspect.

Preferably, the aromatic residue is as defined in the first aspect. Hence, the monomer comprising an aromatic residue may comprise an (S)—N-(1-phenylethyl) (Nspe) glycine monomer, an (R)—N-(1-phenylethyl) glycine (Nrpe) monomer, an N-(phenylmethyl) glycine (Nphe) monomer, an N-(4-fluoro phenylmethyl) glycine (Npfb) monomer, an N-(3-fluoro phenylmethyl) glycine (Nmfb) monomer, an (S)—N-1-(4-fluoro phenylethyl) glycine (Nsfb) monomer, an (R)—N-1-(4-fluoro phenylethyl) glycine (Nrfb) monomer, an N-(3,5 difluoro phenylmethyl) glycine (Ndfb) monomer, an N-(4-chloro phenylmethyl) glycine (Npcb) monomer, an N-(4-methoxyphenylmethyl) glycine (Npmb) monomer, an N-(methylimidazole) glycine (NHis) monomer, an N-(methylindole) glycine (NTrp) monomer, an N-(4-hydroxy phenylmethyl) glycine (NTyr) monomer, an N-(4-pyridinylmethyl) glycine (NPyr) monomer, an (S)—N-(1-naphthlethyl) glycine (Nsna) monomer, an (R)—N-(1-naphthlethyl) glycine (Nrna) monomer, an N-(furanylmethyl) glycine (Nfur) monomer, an N-(thiofuranylmethyl) glycine (Ntfur) monomer or an N-(diphenylmethyl) glycine (Ndpa) monomer.

The peptoid, analogue or derivative thereof may comprise at least one monomer comprising an aliphatic residue. The monomer comprising an aliphatic residue may be as defined in the first aspect.

In one embodiment the peptoid, analogue or derivative thereof comprises a linear peptoid, analogue or derivative thereof.

The linear peptoid, analogue or derivative thereof may comprise at least 3, 4, 5 or 6 monomers. Preferably, the linear peptoid, analogue or derivative thereof comprises at least 7, 8, 9, 10 or 11 monomers. Most preferably, the linear peptoid, analogue or derivative thereof comprises at least 12 monomers.

The linear peptoid, analogue or derivative thereof may comprise between 3 and 30 monomers. Preferably, the linear peptoid, analogue or derivative thereof comprises between 5 and 20 monomers. Most preferably, the linear peptoid, analogue or derivative thereof comprises between 8 and 15 monomers.

The linear peptoid, analogue or derivative thereof may have the structure (NLys-Nspe-Nspe)₂(NhArg-Nspe-Nspe)₂; (NhArg-Nspe-Nspe)₂(NLys-Nspe-Nspe)₂; (NLys-Nspe-Nspe)(NhArg-Nspe-Nspe) (NLys-Nspe-Nspe)₂; [(NhArg-Nspe-Nspe)(NLys-Nspe-Nspe)]₂; or [(NnArgNspeNspe)(NaeNspeNspe)]₂.

In an alternative embodiment, the peptoid, analogue or derivative thereof comprises a cyclic peptoid, analogue or derivative thereof.

The cyclic peptoid, analogue or derivative thereof may comprise at least 3 monomers. Preferably, the cyclic peptoid, analogue or derivative thereof comprises at least 4 or 5 monomers. Most preferably, the cyclic peptoid, analogue or derivative thereof comprises at least 6 monomers.

The cyclic peptoid, analogue or derivative thereof may comprise between 3 and 30 monomers. Preferably, the cyclic peptoid, analogue or derivative thereof comprises between 4 and 20 monomers. Most preferably, the cyclic peptoid, analogue or derivative thereof comprises between 5 and 10 monomers.

The cyclic peptoid, analogue or derivative thereof may have the structure (NLys-Nphe-NhArg-Nphe-NLys-Nphe).

In accordance with a third aspect, there is provided a cyclic peptoid, analogue or derivative thereof, comprising at least one arginine type monomer and at least one lysine type monomer.

Preferably, the cyclic peptoid, analogue or derivative thereof is obtained using the method of the first aspect.

The cyclic peptoid, analogue or derivative thereof may comprise at least one monomer comprising an aromatic residue. Preferably, the or each monomer comprising an aromatic residue is as defined in the first aspect.

The cyclic peptoid, analogue or derivative thereof may comprise at least one aliphatic residue. The aliphatic residue may be as defined in the first aspect.

The cyclic peptoid, analogue or derivative thereof may comprise at least 3 monomers. Preferably, the cyclic peptoid, analogue or derivative thereof comprises at least 4 or 5 monomers. Most preferably, the cyclic peptoid, analogue or derivative thereof comprises at least 6 monomers.

The cyclic peptoid, analogue or derivative thereof may comprise between 3 and 30 monomers. Preferably, the cyclic peptoid, analogue or derivative thereof comprises between 4 and 20 monomers. Most preferably, the cyclic peptoid, analogue or derivative thereof comprises between 5 and 10 monomers.

The inventors believe that peptoids made in accordance with the present invention may be used as medicaments.

Therefore, in accordance with a fourth aspect there is provided a peptoid, analogue or derivative thereof according to the second or third aspect, for use as a medicament.

In accordance with a fifth aspect, there is provided a peptoid, analogue or derivative thereof according to the second or third aspect, for use in treating, ameliorating or preventing a microbial infection.

In an sixth aspect, there is provided a method of treating, ameliorating or preventing a microbial infection in a subject, the method comprising, administering to a subject in need of such treatment, a therapeutically effective amount of a peptoid, analogue or derivative thereof according to the second or third aspect.

Examples 3 to 5 summarise the surprising antibacterial activity of the peptoids synthesised using methods of the invention.

Accordingly, the microbial infection may comprise a bacterial infection, a fungal infection or a parasitic infection.

In one embodiment, the microbial infection comprises a bacterial infection. The bacterial infection may comprise a gram positive bacterial infection or a gram negative bacterial infection. The bacterium may be from the Escherichia genus, preferably E. coli. The bacterium may be from the Pseudomonas genus, preferably P. aeruginosa. The bacterium may be from the Staphylococcus genus, preferably S. aureus. The bacterium may be from the Serratia genus, preferably S. marcesens.

In one embodiment, the microbial infection comprises a fungal infection. The fungus may be from the Candida genus, preferably C. albicans.

In one embodiment, the microbial infection comprises a parasitic infection. The parasite may be a protozoan parasite.

The parasite may be from the Leishmania genus, preferably L. mexicana or L. donovani. Accordingly, the peptoid, analogue or derivative thereof may be for use in treating, ameliorating or preventing leishmaniasis, preferably cutaneous leishmaniasis or visceral leishmaniasis.

The parasite may be from the Trypanosoma genus. The parasite may be T. brucei, preferably T. brucei rhodesiense. Accordingly, the peptoid, analogue or derivative thereof may be for use in treating, ameliorating or preventing African trypanosomiasis.

It will be appreciated that African trypanosomiasis is known as African sleeping sickness in humans. Accordingly, the peptoid, analogue or derivative thereof may be for use in treating, ameliorating or preventing African sleeping sickness. Alternatively, the parasite may be T. cruzi. Accordingly, the peptoid, analogue or derivative thereof may be for use in treating, ameliorating or preventing Chagas disease.

The parasite may be from the Plasmodium genus, preferably P. falciparum. Accordingly, the peptoid, analogue or derivative thereof may be for use in treating, ameliorating or preventing malaria.

It will be appreciated that peptoids, derivatives or analogues thereof described herein may be used in a medicament which may be used in a monotherapy (i.e. use of the compound alone), for treating, ameliorating, or preventing a microbial infection. Alternatively, the peptoids, derivatives or analogues thereof described herein may be used as an adjunct to, or in combination with, known therapies for treating, ameliorating, or preventing a microbial infection.

The peptoids, derivatives or analogues thereof described herein may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the peptoids, derivatives or analogues thereof described herein may be used in a number of ways. For instance, oral administration may be required, in which case the compound may be contained within a composition that may, for example, be ingested orally in the form of a tablet, capsule or liquid. Compositions comprising the compounds of the invention may be administered by inhalation (e.g. intranasally). Compositions may also be formulated for topical use. For instance, creams or ointments may be applied to the skin.

Compounds according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with compounds used according to the invention is required and which would normally require frequent administration (e.g. at least daily injection).

In a preferred embodiment, peptoid, derivative or analogue thereof may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the peptoid, derivative or analogue thereof that is required is determined by its biological activity and bioavailability, which in turn depends on the mode of administration, the physiochemical properties of the compound, and whether it is being used as a monotherapy, or in a combined therapy. The frequency of administration will also be influenced by the half-life of the compound within the subject being treated. Optimal dosages to be administered may be determined by those skilled in the art, and will vary with the particular compound in use, the strength of the pharmaceutical composition, the mode of administration, and the advancement of the microbial infection. Additional factors depending on the particular subject being treated will result in a need to adjust dosages, including subject age, weight, gender, diet, and time of administration.

Generally, a daily dose of between 0.01 μg/kg of body weight and 500 mg/kg of body weight of the peptoid, derivative or analogue thereof may be used for treating, ameliorating, or preventing a microbial infection depending upon which peptoid is used. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

The peptoid, derivative or analogue thereof may be administered before, during or after onset of microbial infection to be treated. Daily doses may be given as a single administration (e.g. a single daily injection). Alternatively, the microbial infection may require administration twice or more times during a day. As an example, a compound according to the second or third aspect may be administered as two (or more depending upon the severity of the microbial infection being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the compounds according to the invention to a patient without the need to administer repeated doses.

Known procedures, such as those conventionally employed by the pharmaceutical industry (e.g. in vivo experimentation, clinical trials, etc.), may be used to form specific formulations comprising the peptoid, derivative or analogue thereof and precise therapeutic regimes (such as daily doses of the compounds and the frequency of administration). The inventors believe that they are the first to describe a pharmaceutical composition for treating a microbial infection, based on the use of the peptoid, derivative or analogue thereof according to the invention.

Hence, in a seventh aspect of the invention, there is provided a pharmaceutical composition comprising a peptoid, analogue or derivative thereof according to the second or third aspect, and a pharmaceutically acceptable vehicle.

The pharmaceutical composition can be used in the therapeutic amelioration, prevention or treatment in a subject of a microbial infection. Thus, the composition is preferably an antimicrobial pharmaceutical composition. Most preferably, the composition is an antibacterial composition.

The invention also provides, in an eighth aspect, a process for making the composition according to the seventh aspect, the process comprising contacting a therapeutically effective amount of a peptoid, analogue or derivative thereof according to the second or third aspect and a pharmaceutically acceptable vehicle.

A “subject” may be a vertebrate, mammal, or domestic animal. Hence, compounds, compositions and medicaments according to the invention may be used to treat any mammal, for example livestock (e.g. a horse), pets, or may be used in other veterinary applications. Most preferably, however, the subject is a human being.

A “therapeutically effective amount” of a peptoid, analogue or derivative thereof according to the second or third aspect is any amount which, when administered to a subject, is the amount of drug that is needed to treat the target disease, or produce the desired effect, i.e. prevent or reduce the microbial infection.

For example, the therapeutically effective amount of peptoid, analogue or derivative thereof used may be from about 0.01 mg to about 800 mg, and preferably from about 0.01 mg to about 500 mg. It is preferred that the amount of compound is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the compounds described herein) according to the invention. In tablets, the active compound may be mixed with a vehicle having the necessary compression properties in suitable proportions and compacted in the shape and size desired. The powders and tablets preferably contain up to 99% of the active compound. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compounds according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound may be prepared as a sterile solid composition that may be dissolved or suspended at the time of administration using sterile water, saline, or other appropriate sterile injectable medium.

The compound and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compounds used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows the comparative structures of an exemplary peptide and a corresponding exemplary peptoid;

FIG. 2 shows a prior art reaction sequence for synthesising guanidinylate peptoids;

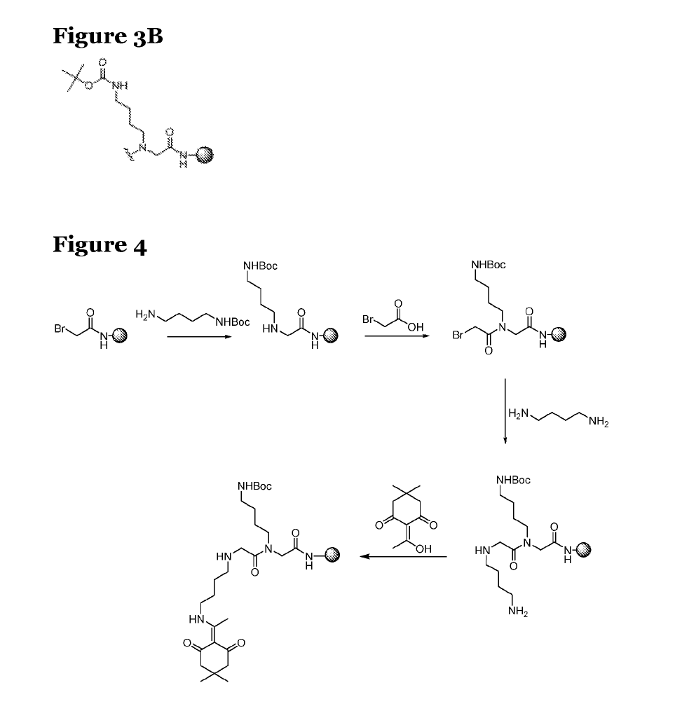

FIG. 3A shows a Dde-protected NLys residue used in a method in accordance with an embodiment of the invention;

FIG. 3B shows a Boc-protected NLys residue used in a method in accordance with an embodiment of the invention;

FIG. 4 is a reaction scheme according to an embodiment of the invention showing how the Dde-protected NLys residue of FIG. 3A can be synthesised and incorporated into an extended peptoid chain;

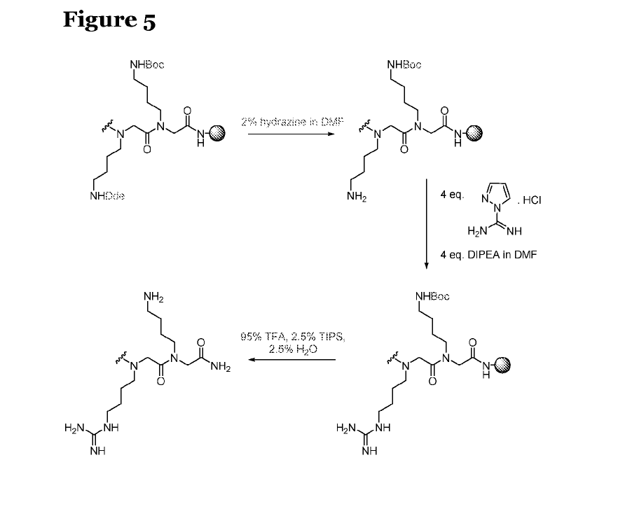

FIG. 5 is a reaction scheme according to another embodiment of the invention showing how a linear mixed peptoid can be synthesised;

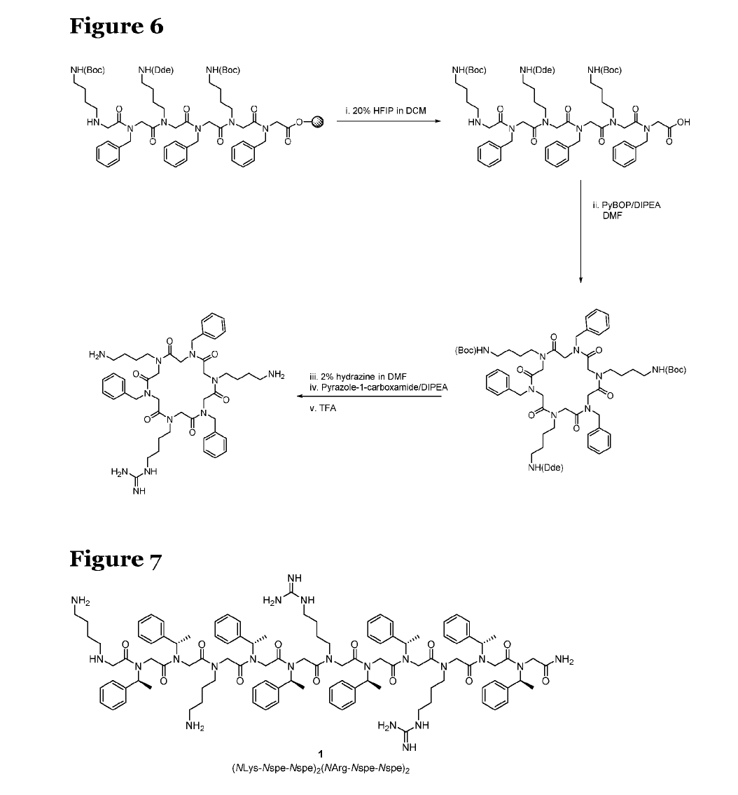

FIG. 6 is a reaction scheme according to another embodiment of the invention showing how a cyclic mixed peptoid can by synthesised;

FIG. 7 shows the structure of a first embodiment of a peptoid referred to herein as “peptoid 1”;

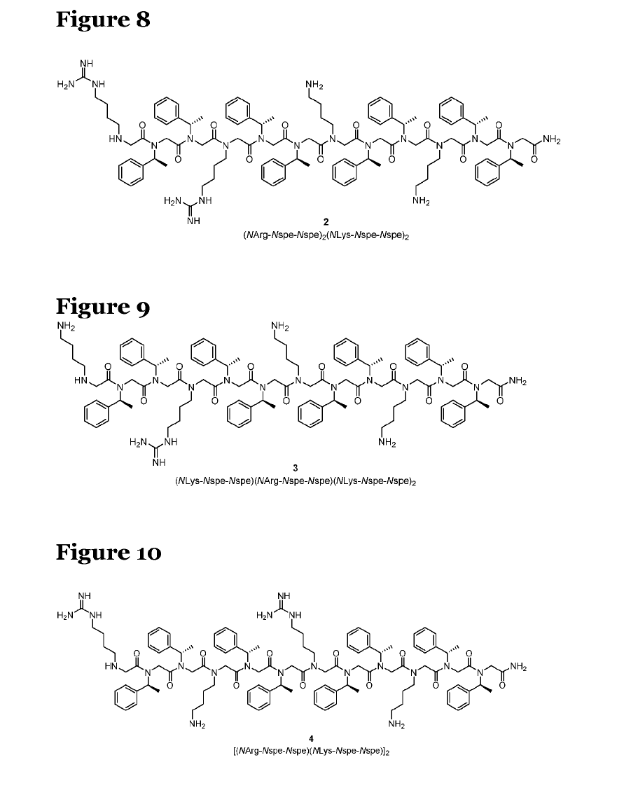

FIG. 8 shows the structure of a second embodiment of a peptoid referred to herein as “peptoid 2”;

FIG. 9 shows the structure of a third embodiment of a peptoid referred to herein as “peptoid 3”;

FIG. 10 shows the structure of a fourth embodiment of a peptoid referred to herein as “peptoid 4”;

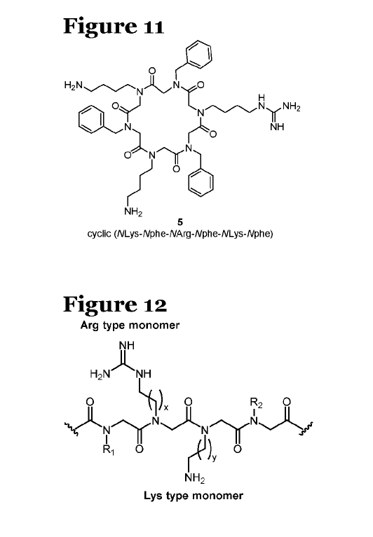

FIG. 11 shows the structure of a fifth embodiment of a peptoid referred to herein as “peptoid 5”;

FIG. 12 is a generic structure for a peptoid in accordance with the present invention;

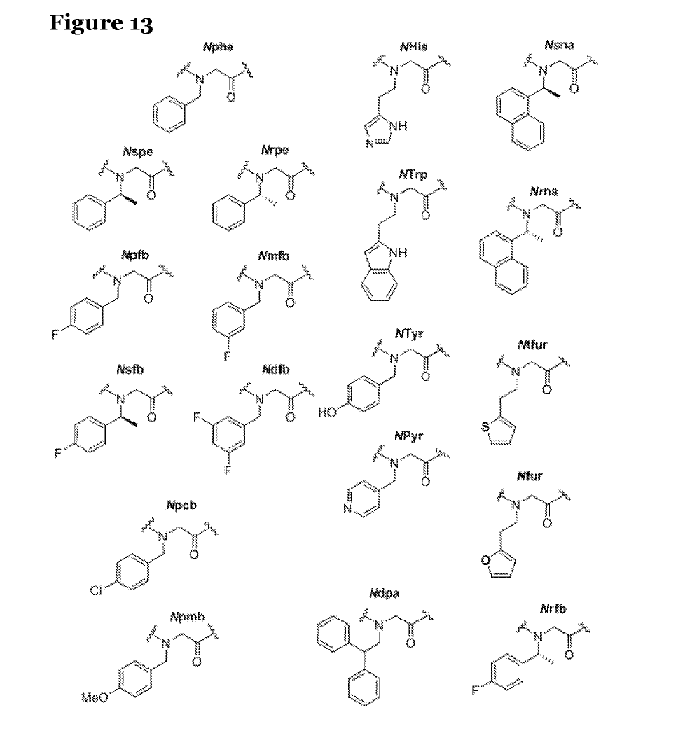

FIG. 13 shows monomers comprising aromatic residues which may comprise part of a peptoid of the present invention;

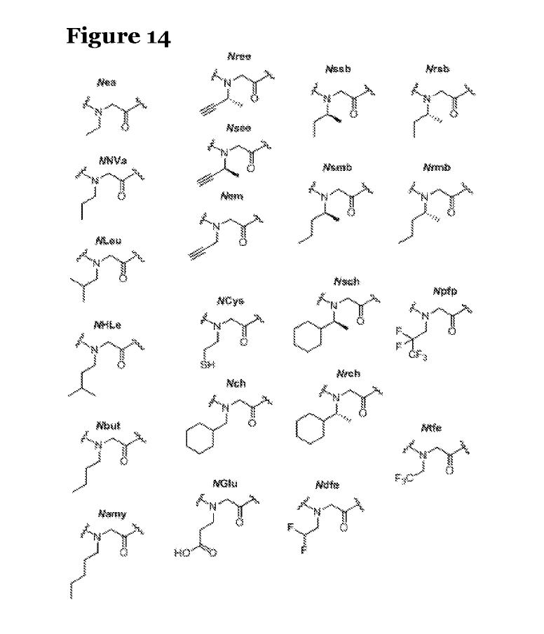

FIG. 14 shows monomers comprising aliphatic residues which may comprise part of a peptoid of the present invention;

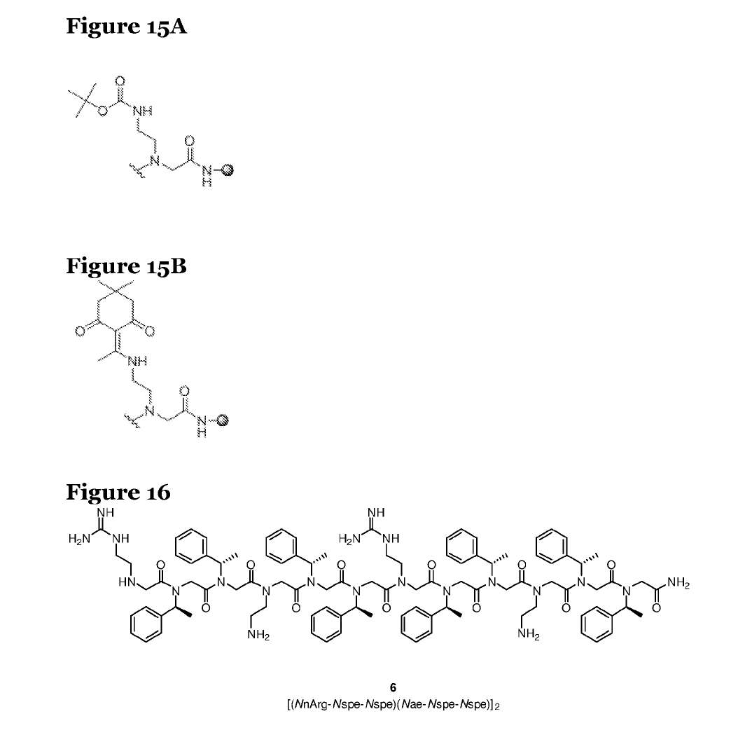

FIG. 15A shows a Dde-protected Nae residue used in a method in accordance with an embodiment of the invention;

FIG. 15B shows a Boc-protected Nae residue used in a method in accordance with an embodiment of the invention;

FIG. 16 shows the structure of a sixth embodiment of a peptoid referred to herein as “peptoid 6”;

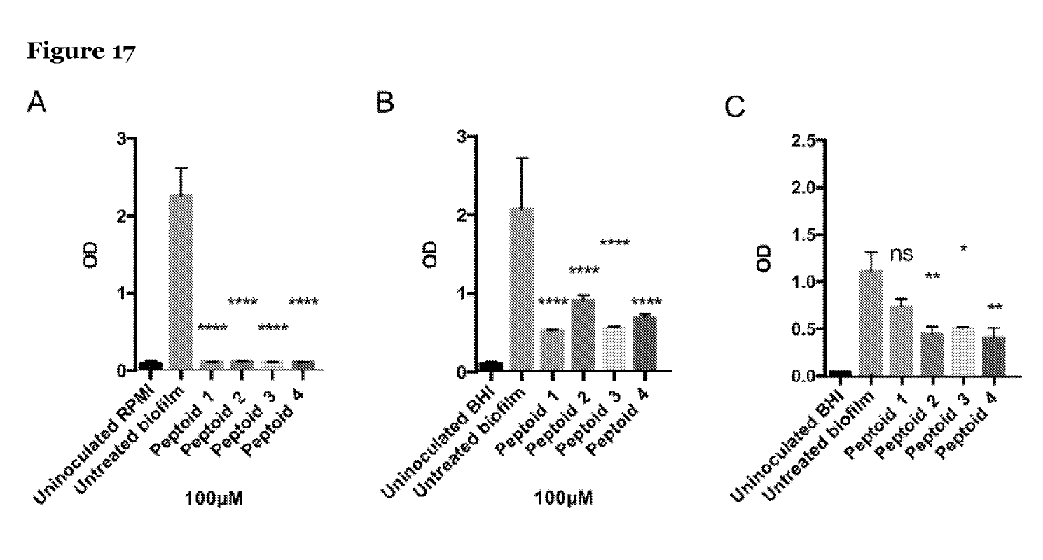

FIG. 17A is a graph showing the activity of peptoids 1-4 against C. albicans;

FIG. 17B is a graph showing the activity of peptoids 1-4 against S. aureus; and

FIG. 17C is a graph showing the activity of peptoids 1-4 against E. coli.

### EXAMPLE 1—SYNTHESIS OF LINEAR MIXED PEPTOIDS

The inventors have developed a novel method using orthogonal protecting groups to protect lysine type residues, such as NLys residues and Nae residues, to synthesise linear mixed peptoids containing lysine type resides, arginine type residues and aromatic side chains.

Materials and Methods

Procedure 1—on Resin Peptoid Synthesis

Fmoc-protected Rink Amide resin (normally 100-300 mg, 0.1-0.3 mmol, typical loading between 0.6-0.8 mmol g-1) was swollen in DMF (at least 1 hour, overnight preferred, at room temperature) in a 20 mL polypropylene syringe fitted with two polyethylene frits (Crawford Scientific). The resin was deprotected with piperidine (20% in DMF v/v, 2×20 min) and washed with DMF (3×2 mL). The resin was treated with bromoacetic acid (1 ml, 0.6M in DMF) and DIC (0.18 ml, 50% v/v in DMF) for 20 minutes at room temperature at 400 rpm on a shaker platform. The resin was washed with DMF (3×2 mL), before the desired amine sub-monomer was added (1 ml, 0.8-2M in DMF) and allowed to react for 60 minutes at room temperature on the shaker. The resin was again washed with DMF (3×2 mL).

For instance, when the desired monomer was a Boc protected NLys monomer the desired amine sub-monomer was N-Boc 1,4 diaminobutane, as shown in step 1 of FIG. 4. Alternatively, when the desired monomer unit was a Boc protected Nae monomer unit the desired amine sub-monomer was N-Boc 1,2 diaminoethane.

When the desired monomer was a Dde protected NLys monomer the desired amine sub-monomer was 1,4 diaminobutane, as shown in step 3 of FIG. 4. Alternatively, when the desired monomer unit was a Dde protected Nae monomer unit the desired amine sub-monomer was 1,2 diaminoethane. In both these cases procedure 2 would then be conducted to add the Dde protecting group before procedure 1 was repeated.

When the desired monomer was Nspe the amine sub-monomer was (S)-(−)-α-methylbenzylamine.

Procedure 1 was repeated until the desired peptoid sequence had been obtained. Once the desired full length orthogonally protected peptoid had been obtained procedures 3 to 5 were undertaken sequentially.

Procedure 2—Dde Protection of NLys Submonomer

Dde-OH (10 eq. wrt resin, 1M in DMF) was added to the resin and placed on the shaker at RT for 60 minutes, then the resin was washed well with DMF (3×2 mL).

As explained above, subsequent peptoid couplings were then made by repeating procedure 1 until the desired full length peptoid sequence was obtained.

Procedure 3—Guanidinylation of the Free Amines

On resin deprotection of the Dde group was undertaken using 2% hydrazine in DMF (4×4 ml×3 mins) and then the resin washed with DMF (3×2 mL). Guanidinylation of the free amines were undertaken using pyrazole-1-carboxamide (6 eq. per free amine, in the minimum amount of DMF) and DIPEA (6 eq. per free amine) on the shaker at 400 rpm, RT for 90 minutes.

Procedure 4—Cleavage of Peptoid from Substrate

The resin was washed with DMF (3×2 mL) before cleavage. Final cleavage from resin was achieved using 95:2.5:2.5 TFA:H2O:TIPS (4 ml) for 1.5 hours and the resin removed by filtration. The cleavage cocktail was removed in vacuo, the crude product precipitated in diethyl ether (45 mL) and the precipitate retrieved by centrifuge for 15 min at 5,000 rpm. The ether phase was decanted, the crude product dissolved in a mixture of acidified H2O (0.1% TFA) and MeCN and lyophilised.

Procedure 5—Purification of Crude Peptoids

Crude peptoids were dissolved into 1.5 mL (95% H2O, 5% MeCN, 0.1% TFA) and purified by preparative RP-HPLC using a Perkin Elmer 200 Series LC pump with a Perkin-Elmer 785A UV-vis detector (λ=250 nm) on a SB Analytical column (ODS-H Optimal), 250×10 mm, 5 μm; flow rate=2 mL min-1; linear gradient elution 0-50% solvent B over 60 minutes, then 50-100% B over 15 minutes (solvent A=0.1% TFA in 95% H2O, 5% MeCN, solvent B=0.1% TFA in 5% H20, 95% MeCN). Relevant fractions were collected, lyophilized and analysed by LC-MS and analytical RP-HPLC.

All peptoids were obtained with >95% purity.

Results

NLys residues protected with Boc and Dde protecting groups are shown in FIGS. 3A and 3B respectively, and Nae residues protected with Boc and Dde protecting groups are shown in FIGS. 15A and 15B respectively.

For the synthesis of linear peptoids the procedures 1 and 2, given above, were used in combination to assemble the orthogonally protected specific peptoid sequence on a resin, as shown in FIG. 4. Using procedure 3, Dde was then easily removed using 2% hydrazine in DMF which allowed these residues to be selectively deprotected, while leaving Boc protection on other lysine type chains intact, as shown in step 1 of FIG. 5. The deprotected lysine type residues can then selectively undergo guanidinylation to introduce arginine type residues, as shown in step two of FIG. 5. Using procedure 4, the peptoids can then be cleaved from the resin, and the Boc protection group can be removed in one final step to obtain a mixed peptoid containing both lysine type residues and arginine type residues, as shown in step 3 of FIG. 5. Finally, using procedure 5, the peptoids can be purified.

Five different embodiments of linear peptoids, referred to as peptoids 1 to 4 and 6, were prepared using the above methodology. The structures of the peptoids are shown in FIGS. 7 to 10 and 16, and are:

Peptoid 1 has the structure (NLys-Nspe-Nspe)₂(NhArg-Nspe-Nspe)₂—[SEQ ID No:1];

Peptoid 2 has the structure (NhArg-Nspe-Nspe)₂(NLys-Nspe-Nspe)₂—[SEQ ID No:2];

Peptoid 3 has the structure (NLys-Nspe-Nspe)(NhArg-Nspe-Nspe)(NLys-Nspe-Nspe)₂—[SEQ ID No:3];

Peptoid 4 has the structure [(NhArg-Nspe-Nspe)(NLys-Nspe-Nspe)]₂—[SEQ ID No:4];

Peptoid 6 has the structure [(NnArgNspeNspe)(NaeNspeNspe)]₂—[SEQ ID No:6].

All of the peptoids are 12 residue linear peptoids. Peptoids 1, 2 and 4 each contain two lysine type monomers (NLys), two arginine type monomers (NhArg) and eight aromatic residues (Nspe i.e. (S)—N-1-phenylethyl), as shown in FIGS. 7, 8 and 10.

Peptoid 3 contains three lysine type monomers (NLys), one arginine type monomer (NhArg) and eight aromatic residues (Nspe i.e. (S)—N-1-phenylethyl), as shown in FIG. 9.

Peptoid 6 contains two lysine type monomers (Nae) and two arginine type monomers (NnArg).

Conclusion

The inventors have found that, by using orthogonal protecting groups to protect lysine type residues, it is possible to synthesise linear peptoids containing lysine type residues, arginine type residues and aromatic side chains. This has not been possible previously.

While Nspe is used as an additional monomer in this example it will be appreciated that further monomers could be used. Examples of appropriate monomers comprising aromatic residues are shown in FIG. 13 and examples of appropriate monomers comprising aliphatic residues are shown in FIG. 14.

The orthogonal protecting groups used in this example are Boc and Dde. However, it will be appreciated that alternative protecting groups could be used.

The method devised by the inventors is a versatile method allowing the Dde-protecting group to be added and selectively deprotected in a variety of positions in the peptoid sequence, both near C and N-terminal positions and also within close proximity to each other.

### EXAMPLE 2—SYNTHESIS OF CYCLIC MIXED PEPTOIDS

The inventors have developed a novel method using orthogonal protecting groups to synthesise cyclic mixed peptoids comprising lysine and arginine side chains.

Materials and Methods

Procedure 1—on Resin Peptoid Synthesis

2-chlorotrityl chloride resin (0.1 mmol, typical loading 1.22 mmol g⁻¹) was swollen in dry DCM (45 mins, at room temperature) in a 20 mL polypropylene syringe fitted with two polyethylene frits. The resin was washed with dry DCM (3×2 mL) and loaded with bromoacetic acid (1 ml, 0.6 M in DMF) and neat DIPEA (16 eq. with respect to the resin) for 30 minutes at RT on a shaker at 400 rpm. The resin was washed with DMF (3×2 mL), before the desired amine sub-monomer was added (1 ml, 1.5 M in DMF) and allowed to react for 60 minutes at RT on the shaker.

For instance, when the desired monomer was a Boc protected NLys monomer the desired amine sub-monomer was N-Boc 1,4 diaminobutane, as shown in step 1 of FIG. 4.

When the desired monomer was a Dde protected NLys monomer the desired amine sub-monomer was 1,4 diaminobutane, as shown in step 3 of FIG. 4. In this case procedure 2 would then be conducted to add the Dde protecting group before procedure 1 was repeated.

When the desired monomer was Nphe the amine was benzylamine.

Procedure 1 was repeated until the desired peptoid sequence had been obtained. Once the desired full length orthogonally protected peptoid had been obtained procedures 3 to 7 were undertaken sequentially.

Procedure 2—Dde Protection of NLys Submonomer

Dde-OH (10 eq. wrt resin, 1M in DMF) was added to the resin and placed on the shaker at RT for 60 minutes, then the resin washed well with DMF (3×2 mL).

As explained above, subsequent peptoid couplings were then made by repeating procedure 1 until the desired full length peptoid sequence was obtained.

Procedure 3—Cleavage of Peptoid from Substrate

Final cleavage from resin was achieved using HFIP (4 mL, 20% v/v in DCM) for 30 minutes. The resin was removed by filtration and the cleavage cocktail sparged off using a fine stream of N₂. The crude product was precipitated in diethyl ether (15 mL) and the precipitate retrieved by centrifuge for 15 min at 5,000 rpm. The ether phase was decanted, the crude, protected product dissolved in a mixture of acidified H₂O (0.1% TFA) and MeCN and lyophilised.

Procedure 4—Cyclisation of Peptoid

The crude peptoid was cyclised in solution without further purification. The linear peptoid (100 μmol) was dissolved in dry DMF (10 mL) and added dropwise to a solution of PyBOP and DIPEA (both 6 eq. with respect to the crude linear peptoid, in 10 mL DMF) over 8 hours. The reaction was allowed to proceed for a further 60 minutes at room temperature following the last addition. The DMF solvent was removed in vacuo and the crude peptoids were extracted using DCM (2×20 mL). The organic phases were combined, washed with water and dried over MgSO₄ before filtration and solvent removal in vacuo. The resulting residue was dissolved in 50% MeCN in H₂O and lyophilised.

The protected peptoids were then dissolved in 50% MeCN in H₂O and purified by preparative RP-HPLC; flow rate=2 mL min⁻¹; injection made at 50% B and a linear gradient elution 50-100 solvent B over 60 minutes (solvent A=0.1% TFA in 95% H₂O, 5% MeCN, solvent B=0.1% TFA in 5% H₂O, 95% MeCN). Relevant fractions were collected, lyophilized and analyzed by LC-MS.

Procedure 5—Guanidinylation of the Free Amines

At this stage, Dde-groups were removed using 2% hydrazine in DMF (4×4 ml×3 mins) and then the resin washed with DMF (3×2 mL). Guanidinylation of the free amines were undertaken using pyrazole-1-carboxamide (6 eq. per free amine, in the minimum amount of DMF) and DIPEA (6 eq. per free amine) on the shaker at 400 rpm, RT for 90 minutes.

Procedure 6—Removal of Boc Protecting Groups

The cyclic peptoids were then Boc-deprotected using 95:2.5:2.5 TFA:H₂O:TIPS (4 ml) for 1.5 hours. The cleavage cocktail was removed in vacuo, the crude product precipitated in diethyl ether (45 mL) and the precipitate retrieved by centrifuge for 15 min at 5,000 rpm. The ether phase was decanted, the crude product dissolved in a mixture of acidified H₂O (0.1% TFA) and MeCN and lyophilised.

Procedure 7—Purification of Crude Peptoids

The peptoids were dissolved in 1.5 mL (95% H₂O, 5% MeCN, 0.1% TFA) and purified by preparative RP-HPLC flow rate=2 mL min⁻¹; linear gradient elution 0-50% solvent B over 60 minutes, then 50-100% B over 15 minutes. Relevant fractions were collected, lyophilized and analyzed by LC-MS and analytical RP-HPLC.

All peptoids were obtained with >95% purity.

Results

Linear precursors were synthesised on 2-chlorotrityl chloride resin using procedures 1 and 2, given above. Again, this reaction sequence will be as shown in FIG. 4. Using procedure 3, the linear precursors were cleaved from the resin without removing either the Boc or the Dde protecting groups, as shown in step 1 of FIG. 6. Using procedure 4, a head-to-tail, solution phase cyclisation was then undertaken, as shown in step 2 of FIG. 6. Following cyclisation, the crude cyclic species was purified via RP-HPLC and then, using procedure 5, the Dde groups could be selectively deprotected and a guanidinylation reaction carried out in solution. Finally, the Boc protecting groups could also be removed, using procedure 6. These two procedures are shown as the final step in FIG. 6. Finally, using procedure 7 RP-HPLC purification was carried out to obtain peptoids with >95% purity.

One cyclic peptoid, referred to as peptoid 5, was prepared using the above methodology. The structure of the peptoid is shown in FIG. 11, and is cyclic (NLys-Nphe-NhArg-Nphe-NLys-Nphe)—[SEQ ID No:5].

Peptoid 5 is a six residue cyclic peptoid containing two lysine type monomers (NLys), one arginine type monomer (NhArg), and three aromatic residues (Nphe).

Conclusion

The synthesis of peptoid 5 shows that by using orthogonal protecting groups, it is possible to synthesise cyclic peptoids comprising lysine and arginine side chains. The inventors believe that this is the first example of a cyclic peptoid that contains an Arg type monomer in combination with a Lys type monomer.

While Nphe is used as an additional monomer in this example it will be appreciated that, as with Example 1, further monomers could be used.

As with example 1, the orthogonal protecting groups used in this example are also Boc and Dde. However, it will be appreciated that alternative protecting groups could be used.

Despite the bulky nature of the Dde group, the cyclisation reaction still occurred efficiently at room temperature and complete cyclisation was possible.

### EXAMPLE 3—BIOLOGICAL DATA: PLANKTONIC BACTERIA

The inventors have demonstrated that the mixed peptoids prepared as described above exhibit surprising antibacterial properties against planktonic bacteria.

Materials and Methods

Bacterial Strains

Species used in MIC assays included gram-negative Escherichia coli K12 W3110, Pseudomonas aeruginosa laboratory strain PAO2 and Serratia marcescens laboratory strain and gram-positive Staphylococcus aureus NCTC 6571 and Micrococcus luteus laboratory strain.

Overnight Culture Preparation

Bacterial cultures were prepared by streaking the bacterial strains on to agar plates with an inoculation loop and incubating overnight at 37° C. A single colony was then selected and placed in 5 mL of Iso-Sensitest broth using an inoculation loop and incubated at 37° C. with shaking overnight.

MIC Determination

MIC values were attained according to the protocol described by J. M. Andrews et al. [J. M. Andrews, J. Antimicrob. Chemother., 2001, 48, 5-16] and were conducted in 96-well microtitre plates in triplicate. 10-50 μL of each overnight culture was inoculated into 1.2 mL of Iso-Sensitest broth and grown at 37° C. with shaking. An inoculum density of ˜10⁴ cfu/spot was determined by comparison with 0.5 MacFarland standard (240 μM BaCl₂ in 0.18 M H₂SO_{4 aq}.) and was found to relate to an A₆₅₀ₙₘ of 0.07 after calibration with regular Iso-Sensitest broth. The inoculum was diluted ten-fold with Iso-Sensitest broth before use (to ˜10³ cfu/spot). Peptide solutions were initially dissolved in DMSO (5 mg mL⁻¹) and then diluted further with Iso-Sensitest broth to achieve a concentration range of 4 mgL⁻¹ to 512 mgL⁻¹ using 2-fold serial dilutions. Samples were vortexed between dilutions where necessary to aid dissolution. 50 μL of inoculum and 50 μL of peptide solution were added to each test well to achieve a final concentration range of 2 mgL⁻¹ to 256 mgL⁻¹. Separate dilutions of ampicillin and DMSO were made up in a similar manner to act as a positive antibacterial control and a +DMSO control, respectively. 50 μL of inoculum and 50 μL of Iso-Sensitest broth were used as a positive control and 100 μL of inoculum was used as a negative control. Positive and negatives controls were conducted multiple times in parallel per plate. MIC was defined as the lowest concentration which completely inhibited bacterial growth after incubation at 37° C. for 16 h with shaking. IC₅₀ was defined as the concentration of antibiotic which achieved a 50% inhibition of bacterial growth after incubation at 37° C. for 16 h with shaking. Quantitative data were attained as A₆₅₀ₙₘ values using a BioTek® Synergy™ H4 Hybrid Multi-Mode Microplate Reader.

Results

The anti-bacterial activity of peptoids 1 to 4 are shown in Table 1.

TABLE 1
Anti-bacterial properties of peptoids 1 to 4

| Pep- | MIC (μM/mgL⁻¹) | | | |
|---|---|---|---|---|
| toid | E. coli | P. aeruginosa | S. aureus | S. marcescens |
| 1 | 17/32 | 34/64 | 17/32 | 134/256 |
| 2 | 17/32 | 17/32 | 17/32 | 134/256 |
| 3 | 17/32 | 17/32 | 17/32 | 134/256 |
| 4 | 17/32 | 67/128 | 17/32 | >134/>256 |

Conclusion

The mixed Arg/Lys type monomer containing peptoids (1-4) have been shown to have anti-bacterial properties against both Gram positive and Gram negative bacteria.

### EXAMPLE 4—BIOLOGICAL DATA: BIOFILM DATA

The inventors have demonstrated that the mixed peptoids prepared as described above exhibit surprising antibacterial and antifungal properties against bacterial and fungal biofilms.

Materials and Methods

Micro-Organism Strains and Growth Conditions

C. albicans (NCTC 3179) was subcultured aerobically on Sabouraud agar plates and propagated in yeast peptone dextrose broth. E. coli (ATCC 29522) and S. aureus (NCTC 6571) were grown on blood agar plates and propagated in brain heart infusion (BHI) broth.

Preparation and Treatment of Single Species Biofilms

Overnight cultures of C. albicans were washed and resuspended in a modified RPMI-1640 (Sigma-Aldrich, St Louis, USA) medium to yield an inoculum of 1.0×10⁶ cells/ml. Overnight cultures of S. aureus or E. coli were washed and resuspended in brain heart infusion broth (Oxoid, Basingstoke, UK) to yield an inoculum of 5.0×10⁶ cells/ml. A total volume of 100 μl of each inoculum was added to microtitre plate wells (Thermo Fisher Scientific, Roskilde, Denmark). An initial biofilm was allowed to form for 4 hours. Wells were washed three times with 200 μl PBS to facilitate removal of planktonic cells and the biofilms were then treated with 100 μM of peptoids 1-4 in the appropriate broth. Plates were incubated for a further 24 hours to allow biofilm maturation. After removal of planktonic cells by washing, biofilms were quantified by the crystal violet assay or by PMA-qPCR.

Biofilm quantification by crystal violet assay Washed biofilms were fixed with 100 μl methanol for 10 minutes. Following removal of methanol, the wells were air dried and stained with crystal violet solution (Clin-Tech Ltd, Guildford, UK) for 20 minutes at room temperature. Excess stain was removed by washing, the plate was then air dried and bound crystal violet was re-solubilised in 16 μl 33% acetic acid prior to reading at 570 nm in a microtitre plate reader (Tecan GENios, Ziirich, Switzerland).

Results

As shown in FIG. 17A, all of the peptoids 1 to 4 exhibited strong antifungal activity against the C. albicans biofilm. Furthermore, as shown in FIG. 17B, all of peptoids 1 to 4 exhibited strong anti-bacterial activity against the S. aureus biofilm, with peptoids 1 and 3 exhibiting the strongest anti-bacterial activity. Finally, as shown in FIG. 17C, all of peptoids 1 to 4 exhibited anti-bacterial activity against the E. coli biofilm, with peptoids 2 and 4 exhibiting the strongest anti-bacterial activity

Conclusion

The mixed Arg/Lys type monomer containing peptoids (1-4) have been shown to have antimicrobial properties against fungus (C. albicans), gram positive bacteria (S. aureus) and gram negative bacteria (E. coli).

### EXAMPLE 5—BIOLOGICAL DATA: ANTI-PARASITIC ACTIVITY

The inventors have demonstrated that the mixed peptoids prepared as described above exhibit surprising antiparasitic activity against clinically relevant parasites that cause various diseases; malaria, Chagas disease, African sleeping sickness and Leishmaniasis.

Materials and Methods

Activity against Trypanosoma brucei rhodesiense STIB900.

This stock was isolated in 1982 from a human patient in Tanzania and after several mouse passages cloned and adapted to axenic culture conditions. Minimum Essential Medium (50 μl) supplemented with 25 mM HEPES, 1 g/l additional glucose, 1% MEM non-essential amino acids (100×), 0.2 mM 2-mercaptoethanol, 1 mM Na-pyruvate and 15% heat inactivated horse serum was added to each well of a 96-well microtiter plate. Serial drug dilutions of eleven 3-fold dilution steps covering a range from 100 to 0.002 μg/ml were prepared. Then 4×10³ bloodstream forms of T. b. rhodesiense STIB 900 in 50 μl was added to each well and the plate incubated at 37° C. under a 5% CO₂ atmosphere for 70 h. 10 μl Alamar Blue (resazurin, 12.5 mg in 100 ml double-distilled water) was then added to each well and incubation continued for a further 2-4 h. Then the plates were read with a Spectramax Gemini XS microplate fluorometer (Molecular Devices Cooperation, Sunnyvale, Calif., USA) using an excitation wave length of 536 nm and an emission wave length of 588 nm. The IC50 values were calculated by linear regression from the sigmoidal dose inhibition curves using SoftmaxPro software (Molecular Devices Cooperation, Sunnyvale, Calif., USA). Melarsoprol (Arsobal Sanofi-Aventis, received from WHO) is used as control.

Activity Against T. cruzi.

Rat skeletal myoblasts (L-6 cells) were seeded in 96-well microtitre plates at 2000 cells/well in 100 μL RPMI 1640 medium with 10% FBS and 2 mM 1-glutamine. After 24 h the medium was removed and replaced by 100 μl per well containing 5000 trypomastigote forms of T. cruzi Tulahuen strain C2C4 containing the β-galactosidase (Lac Z) gene. After 48 h the medium was removed from the wells and replaced by 100 μl fresh medium with or without a serial drug dilution of eleven 3-fold dilution steps covering a range from 100 to 0.002 μg/ml. After 96 h of incubation the plates were inspected under an inverted microscope to assure growth of the controls and sterility. Then the substrate CPRG/Nonidet (50 μl) was added to all wells. A color reaction developed within 2-6 h and could be read photometrically at 540 nm. Data were analyzed with the graphic programme Softmax Pro (Molecular Devices), which calculated IC₅₀ values by linear regression from the sigmoidal dose inhibition curves. Benznidazole is used as control (IC50 0.5+0.2 μg/ml).

Activity Against L. donovani Axenic Amastigotes

Amastigotes of L. donovani strain MHOM/ET/67/L82 were grown in axenic culture at 37° C. in SM medium²⁴ at pH 5.4 supplemented with 10% heat-inactivated fetal bovine serum under an atmosphere of 5% CO₂ in air. One hundred microlitres of culture medium with 10⁵ amastigotes from axenic culture with or without a serial drug dilution were seeded in 96-well microtitre plates. Serial drug dilutions of eleven 3-fold dilution steps covering a range from 90 to 0.002 μg/ml were prepared. After 70 h of incubation the plates were inspected under an inverted microscope to assure growth of the controls and sterile conditions. 10 μl of Alamar Blue (12.5 mg resazurin dissolved in 100 ml distilled water) were then added to each well and the plates incubated for another 2 h. Then the plates were read with a Spectramax Gemini XS microplate fluorometer (Molecular Devices Cooperation, Sunnyvale, Calif., USA) using an excitation wave length of 536 nm and an emission wave length of 588 nm. Data were analyzed using the software Softmax Pro (Molecular Devices Cooperation, Sunnyvale, Calif., USA). Decrease of fluorescence (=inhibition) was expressed as percentage of the fluorescence of control cultures and plotted against the drug concentrations. From the sigmoidal inhibition curves the IC₅₀ values were calculated.

Activity Against P. falciparum.

In vitro activity against erythrocytic stages of P. falciparum was determined using a 3H-hypoxanthine incorporation assay using the drug sensitive NF54 strain (Schipol airport) or the chloroquine and pyrimethamine resistant K1 strain that originate from Thailand and the standard drug chloroquine (Sigma C6628). Compounds were dissolved in DMSO at 10 mg/ml and added to parasite cultures incubated in RPMI 1640 medium without hypoxanthine, supplemented with HEPES (5.94 g/l), NaHCO₃ (2.1 g/1), neomycin (100 U/ml), Albumax® (5 g/l) and washed human red cells A⁺ at 2.5% haematocrit (0.3% parasitaemia). Serial drug dilutions of eleven 3-fold dilution steps covering a range from 100 to 0.002 μg/ml were prepared. The 96-well plates were incubated in a humidified atmosphere at 37° C.; 4% CO₂, 3% O₂, 93% N₂. After 48 h 50 μl of 3H-hypoxanthine (=0.5 μCi) was added to each well of the plate. The plates were incubated for a further 24 h under the same conditions. The plates were then harvested with a Betaplate™ cell harvester (Wallac, Zurich, Switzerland), and the red blood cells transferred onto a glass fibre filter then washed with distilled water. The dried filters were inserted into a plastic foil with 10 ml of scintillation fluid, and counted in a Betaplate™ liquid scintillation counter (Wallac, Zurich, Switzerland). IC₅₀ values were calculated from sigmoidal inhibition curves by linear regression using Microsoft Excel. Chloroquine and artemisinin are used as control.

Cell Culture of Leishmania Mexicana M379 Promastigotes and Amastigotes

Leishmania mexicana (M379) promastigote parasites were maintained at 26° C. in Schneider's Insect medium (Sigma-Aldrich) supplemented with heat-inactivated foetal bovine sera (FBS, 15%; Biosera Ltd). Cells were counted using a Neubauer Improved Haemocytometer. Promastigotes were transformed into axenic amastigotes by a pH and temperature shift as previously described. A culture of recently transformed (three days) promastigotes in the late log phase was transferred into Schneider's Insect medium supplemented with 20% heat-inactivated FBS (pH 5.5) at 5×105 parasites/mL. After 6 days, the parasites were in the metacyclic stage and used for transformation to amastigote-like forms by transfer in the same medium at 32° C. at 5×105 parasites/mL. After additional 5-7 days, the parasites should be in the amastigote stage and be ready for cytotoxicity studies and infections.

Cytotoxicity Assays with L. Mexicana M379 Promastigotes and Amastigotes

Cytotoxicity analyses were performed in 96-well plates (Costar, Fisher Scientific) using Alamar Blue (Invitrogen) for cell viability detection as previously described.

Promastigote and amastigote L. mexicana were pre-incubated with the compounds in triplicate (5 mM stock solutions in DMSO; Amphotericin B was used as a positive control; untreated parasites with DMSO as a negative control) in 50 μl of the corresponding media at 4×106 mL-1 for 1 hour. Afterwards, 40 μl were removed from each well before the addition of 90 μl of the corresponding media, followed by incubation for 24 hours at 4×105 mL-1. Then, 10 μl Alamar Blue solution (Invitrogen) was added to each well for an incubation of 4 hours prior to assessing cell viability using a fluorescent plate reader (Biotek; Ex 560 nm/Em 600 nm). To investigate the effects of serum on the efficacy of the peptoids, the assay described above was modified using serum-free medium for the pre-incubation time. For these assays, the parasites were washed three times in serum-free medium before adding them to the compound solutions. All of the experiments described above were carried out on a minimum of two separate occasions to ensure a robust data set was collected.

Results

The anti-parasitic activity of peptoids 1, 2, 4 and 6 against biofilm are shown in Table 2.

TABLE 2
Anti-parasitic activities of peptoids 1, 2, 4 and 6.

| | IC50 (μM) | | | | |
|---|---|---|---|---|---|
| Pep- | L. | T. brucei | T. | L. | P. |
| toid | mexicana | rhodesiense | cruzi | donovani | falciparum |
| 1 | 37 | 6.73 | 12.35 | 11.14 | 1.50 |
| 2 | 34 | | | | |
| 4 | 37 | 16.70 | 21.50 | 19.95 | 2.72 |
| 6 | | 6.44 | 6.58 | 9.51 | 0.99 |

Peptoids 1, 2 and 4 were all found to be active against L. mexicana, which causes cutaneous leishmaniasis. Additionally, peptoids 1, 4 and 6 were all found to be active against T. brucei rhodesiense, which causes causes African sleeping sickness, T. cruzi which causes Chagas disease, L. donovani which causes visceral leishmaniasis, and P. falciparum, which causes malaria.

Conclusion

Peptoids 1, 2, 4 and 6 have been shown to have anti-parasitic activities against various protozoan parasites.

1. A method of preparing a peptoid, analogue or derivative thereof, comprising at least one arginine type monomer and at least one lysine type monomer, the method comprising:—
(i) synthesising a precursor linear peptoid, analogue or derivative thereof comprising one or more lysine type monomers protected with a first protecting group, and one or more lysine type monomers protected with a second protecting group, wherein the first and second protecting groups are orthogonal;
(ii) removing the first protecting group to reveal one or more unprotected lysine type monomers;
(iii) converting the one or more unprotected lysine type monomers to one or more arginine type monomers; and
(iv) removing the second protecting group to obtain a peptoid, analogue or derivative thereof, comprising at least one arginine type monomer and at least one lysine type monomer.

2. The method according to claim 1, wherein the arginine type monomer comprises a monomer of Formula (I): 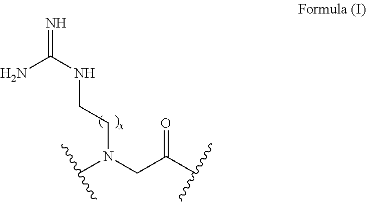
wherein x is an integer between 0 and 14.

3. The method according to claim 1, wherein the lysine type monomer comprises a monomer of Formula (II): 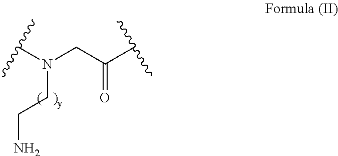
wherein y is an integer between 0 and 14.

4. The method according to claim 1, wherein the first protecting group comprises an N-(1-(4,4-dimethyl-2,6-dioxocyclohexylidene)ethyl) (Dde) protecting group and/or the second protecting group comprises a tert-Butyloxycarbonyl (Boc) protecting group.

5. The method according to claim 1, wherein the step of synthesising the linear precursor peptoid, analogue or derivative thereof comprises:
synthesising a first monomer on a substrate to obtain a linear precursor peptoid, analogue or derivative thereof comprising one monomer;
subsequently adding at least one further monomer in a step-wise fashion to obtain the linear precursor peptoid, analogue or derivative thereof containing the desired number of monomers.

6. The method according to claim 5, wherein the first and/or at least one further monomer comprise at least one monomer comprising an aromatic residue and/or at least one monomer comprising an aliphatic residue, optionally wherein the monomer comprising an aromatic residue comprises an (S)—N-(1-phenylethyl) glycine (Nspe) monomer, an (R)—N-(1-phenylethyl) glycine (Nrpe) monomer, an N-(phenylmethyl) glycine (Nphe) monomer, an N-(4-fluoro phenylmethyl) glycine (Npfb) monomer, an N-(3-fluoro phenylmethyl) glycine (Nmfb) monomer, an (S)—N-1-(4-fluoro phenylethyl) glycine (Nsfb) monomer, an (R)—N-1-(4-fluoro phenylethyl) glycine (Nrfb) monomer, an N-(3,5 difluoro phenylmethyl) glycine (Ndfb) monomer, an N-(4-chloro phenylmethyl) glycine (Npcb) monomer, an N-(4-methoxyphenylmethyl) glycine (Npmb) monomer, an N-(methylimidazole) glycine (NHis) monomer, an N-(methylindole) glycine (NTrp) monomer, an N-(4-hydroxy phenylmethyl) glycine (NTyr) monomer, an N-(4-pyridinylmethyl) glycine (NPyr) monomer, an (S)—N-(1-naphthlethyl) glycine (Nsna) monomer, an (R)—N-(1-naphthlethyl) glycine (Nrna) monomer, an N-(furanylmethyl) glycine (Nfur) monomer, an N-(thiofuranylmethyl) glycine (Ntfur) monomer, or an N-(diphenylmethyl) glycine (Ndpa) monomer, and the monomer comprising an aliphatic residue comprises an N-(pentyl) glycine (Namy) monomer, an N-(propyl) glycine (NNVa) monomer, an N-(isopentyl) glycine (NHLe) monomer, N-(isobutyl) glycine (NLeu) monomer, an N-(butyl) glycine (Nbut) monomer, an N-(2-carboxyethyl) glycine (NGlu) monomer, an N-(2,2,2-trifluoromethyl) glycine (Ntfe) monomer, an N-(2,2,3,3,3-pentafluoropropyl) glycine (Npfp), an N-(2,2-difluoroethyl) glycine (Ndfea) monomer, an N-(ethyl) glycine (Nea) monomer, an N-(2-thioethyl) glycine (NCys) monomer, an (S)—N-(sec-butyl) glycine (Nssb) monomer, an (R)—N-(sec-butyl) glycine (Nrsb) monomer, an (S)—N-(1-methylbutyl) glycine (Nsmb) monomer, an (R)—N-(1-methylbutvl) glycine (Nrmb) monomer, an (S)—N-(1-cyclohexylethyl) glycine (Nsch) monomer, (R)—N-(1-cyclohexylethyl) glycine (Nrch) monomer, an N-(1-cyclohexylmethyl) glycine (Nch) monomer, an N-(ethynylmethyl) glycine (Nem) monomer, an (S)—N-(1-ethynylethyl) glycine (Nsee) monomer, or an (R)—N-(1-ethynylethyl) glycine (Nree) monomer.

7.-8. (canceled)

9. The method according to claim 5, wherein the substrate comprises a resin, optionally Rink amide resin, 2-chlorotrityl chloride resin, Wang resin, 4-(1′,1′-dimethyl-1′-hydroxypropyl) phenoxyacetyl alanyl aminomethyl polystyrene (DHPP) resin or diphenyldiazomethane (PDDM) resin.

10. The method according to claim 5, wherein the method comprises a step of cleaving the peptoid, derivative or analogue thereof from the substrate to obtain a cleaved precursor linear peptoid, analogue or derivative thereof.

11. The method according to claim 10, wherein the cleaving step is carried out subsequent to step (i) and prior to step (ii), optionally wherein the method comprises a cyclisation step comprising cyclising the precursor linear peptoid, derivative or analogue thereof to obtain a precursor cyclic peptoid, wherein the cyclisation step is carried out subsequent to the cleaving step, and prior to step (ii).

12. (canceled)

13. The method according to claim 10, wherein the cleaving step is carried out subsequent to step (iii), optionally wherein the cleaving step is carried out simultaneously to the step of removing the second protecting group.

14. (canceled)

15. A peptoid, analogue or derivative thereof, comprising at least one arginine type monomer, at least one lysine type monomer and at least one monomer comprising an aromatic residue.

16. The peptoid, analogue or derivative thereof according to claim 15, wherein the peptoid, analogue or derivative thereof comprises between 3 and 30 monomers.

17. The peptoid, analogue or derivative thereof according to claim 15, wherein the peptoid, analogue or derivative thereof comprises a linear peptoid, analogue or derivative thereof, optionally wherein the linear peptoid, analogue or derivative thereof has the structure (NLys-Nspe-Nspe)₂(NhArg-Nspe-Nspe)₂; (NhArg-Nspe-Nspe)₂(NLys-Nspe-Nspe)₂; (NLys-Nspe-Nspe)(NhArg-Nspe-Nspe)(NLys-Nspe-Nspe)₂; [(NhArg-Nspe-Nspe)(NLys-Nspe-Nspe)]; or [(NnArgNspeNspe)(NaeNspeNspe)]₂.

18. (canceled)

19. The peptoid, analogue or derivative thereof according to claim 15, wherein the peptoid, analogue or derivative thereof comprises a cyclic peptoid, analogue or derivative thereof, optionally wherein the cyclic peptoid, analogue or derivative thereof has the structure (NLys-Nphe-NhArg-Nphe-NLys-Nphe).

20. (canceled)

21. A cyclic peptoid, analogue or derivative thereof, comprising at least one arginine type monomer and at least one lysine type monomer.

22. (canceled)

23. A method of treating, ameliorating or preventing a microbial infection in a subject, the method comprising, administering to a subject in need of such treatment, a therapeutically effective amount of a peptoid, analogue or derivative thereof according to claim 15.

24. The method according to claim 23, wherein the microbial infection comprises a bacterial infection, optionally a gram positive bacterial infection or a gram negative bacterial infection and/or wherein the bacterium is from the Escherichia genus, preferably E. coli, Pseudomonas genus, preferably P. aeruginosa, Staphylococcus genus, preferably S. aureus, or Serratia genus, preferably S. marcesens.

25.-26. (canceled)

27. The method according to claim 23, wherein the microbial infection comprises a fungal infection, optionally wherein the fungus is from the Candida genus, preferably C. albicans.

28. (canceled)

29. The method according to claim 23, wherein the microbial infection comprises a parasitic infection, optionally wherein the parasite is from the Leishmania genus, preferably L. mexicana or L. donovani, the Trypanosoma genus, preferably T. brucei or T. cruzi, the Plasmodium genus, preferably P. falciparum.

30. (canceled)

31. The method according to claim 29, wherein the method is for a method of treating, ameliorating or preventing leishmaniasis, African trypanosomiasis, Chagas disease or malaria.

32.-33. (canceled)
